SEQUENCE LISTING (1 sequence)

SEQ ID NO: 1 - protein, 4 aa, Homo sapiens
DEVD

(19) United States
(12) Patent Application Publication
(10) Pub. No.: US 2009/0142273 A1
(43) Pub. Date: Jun. 4, 2009

(54) Activatable cest MRI agent

(75) Inventors: Mark Pagel, Shaker Heights, OH (US); Byunghee Yoo, Mayfield Heights, OH (US); Guanshu Liu, Cleveland, OH (US); Rachel Rosenblum, Cleveland, OH (US)

Correspondence Address: MCDONALD HOPKINS LLC, 600 Superior Avenue, East, Suite 2100, CLEVELAND, OH, 44114-2653, US

(73) Assignee: CASE WESTERN RESERVE UNIVERSITY

(21) Appl. No.: 11/811,186

(22) Filed: Jun. 8, 2007

Related U.S. Application Data
(60) Provisional application No. 60/811,852, filed on Jun. 8, 2006.

(51) Int. Cl. A61B 5/055; C12Q 1/02; G01R 33/54

(52) U.S. Cl. 424/9.3; 424/9.36; 424/9.321; 424/9.322; 424/9.341; 435/29; 324/307

(57) ABSTRACT

A chemical exchange saturation transfer (CEST) contrast agent is provided. One embodiment includes a ligand and a functional group linked to the ligand. The functional group has a hydrogen exchange site and is capable of undergoing a change in chemical functionality by enzyme catalysis or reaction with a metabolite to change the chemical exchange rate or the MR frequency of the hydrogen exchange site.

### CROSS-REFERENCE TO RELATED APPLICATIONS

This application claims priority from U.S. Provisional Patent Application No. 60/811,852 entitled “ACTIVATABLE CEST MRI AGENT” filed on Jun. 8, 2006.

### FEDERAL FUNDING NOTICE

The invention was made with federal government support under Federal Grant No. W81XWH-04-1-0731 supplied by the U.S. Army Medical Research and Material Command. The Federal Government has certain rights in the invention.

### COPYRIGHT NOTICE

A portion of the disclosure of this patent document contains material subject to copyright protection. The copyright owner has no objection to the facsimile reproduction of the patent document or the patent disclosure as it appears in the Patent and Trademark Office patent file or records, but otherwise reserves all copyright rights whatsoever.

### BACKGROUND

MRI (magnetic resonance imaging) contrast agents may include a metal atom that causes the excited MR (magnetic resonance) signal of water to relax (e.g., return to an equilibrium or unexcited state). “Relaxivity” may be defined as the rate of water MR signal relaxation per 1 mM of agent. Relaxivity-based MRI contrast agents have been approved for clinical use, and a variety of derivatives have been used in clinical and pre-clinical research to improve pharmacokinetics and disease diagnoses.

A different type of contrast agent, known as “activatable” MRI contrast agents, have been demonstrated to change their relaxivities after being modified by enzymes or metabolic products. Activatable MRI contrast agents may also be referred to as “smart” agents. These activatable MRI contrast agents may detect enzymes and metabolites, however the changes in relaxivities associated with these activatable MRI contrast agents can be relatively insensitive. The detection of “smart” MRI contrast agents based on changes in T1 or T2 relaxivities can be obscured by endogenous changes in T1 or T2 relaxation. Also, changes in relaxation caused by these agents can be relatively modest at high magnetic field strengths. Furthermore, relaxivity-based agents may not be selectively detectable, which inherently limits MRI studies to the detection of a single agent during an MRI scan session.

Chemical Exchange Saturation Transfer (CEST) provides a different method for detecting MRI contrast agents. CEST agents possess a proton exchange site with a unique MR resonance frequency and an appropriate exchange rate with solvent water.

A schematic of activatable CEST MRI agents (including PARACEST MRI AGENTS) is provided in FIG. 1. Step A illustrates a mechanism associated with an activatable CEST MRI agent. Steps B-D illustrate the mechanism of standard CEST agents. MR-detectable hydrogens are associated with number 1. Hydrogens associated with number 2 can not be detected by MRI after a selective MR saturation pulse is applied. During step A, a chemical functional group changes from X to Y. Chemical functional group Y possesses a hydrogen atom that has a unique MR frequency and that can be exchanged with water. X may or may not have a hydrogen atom. If X has a hydrogen atom, then it has a different ¹H MR frequency relative to Y or has a different water exchange rate. X is a chemical that can be changed to a different chemical Y by the presence of a catalyst or reactant in the molecular environment in which the agent R finds itself. Accordingly, during step B, the hydrogen of Y is saturated so that its MR properties are incoherent or “scrambled,” and therefore are not detectable. During step C, the saturated hydrogen of Y exchanges with water, which decreases the amount of MR-detectable water hydrogens. During step D, the amount of water is detected using standard MRI methods. Steps B-D can be repeated to improve detection sensitivity.

Accordingly, saturation of the resonance frequency of the CEST exchange site step B, followed by exchange with solvent water step C, reduces the MR image intensity of the solvent water step D. CEST is an alternative to T1 and T2 contrast mechanisms. CEST MRI agents possess a hydrogen proton with an appropriate exchange rate with water. Saturation of the MR frequency of this proton, followed by exchange with solvent water, reduces the MR signal of the water.

PARACEST (PARAmagnetic CEST) agents incorporate a paramagnetic lanthanide ion and exhibit a range of resonance frequencies that accommodate different exchange rates. The paramagnetic lanthanide ion shifts the MR frequency of the exchangeable proton to unique values to facilitate selective detection. Endogenous MR contrast can be continually monitored by not saturating the MR frequency of the exchangeable proton. Also, PARACEST agents can be designed with good detection sensitivities.

PARACEST facilitates providing molecular-scale information using MR imaging. Measurements of tissue pH, temperature, glucose concentrations and metabolite levels have been accomplished by detecting the PARACEST effect of exogenous agents that chelate lanthanide ions. However, the modest sensitivity of PARACEST agents, often requiring a minimum concentration of 1-10 mM for adequate detection, has limited the applicability of this approach to detect endogenous molecular targets that only exist at high concentrations within tissues.

### BRIEF DESCRIPTION OF THE DRAWINGS

FIG. 1 illustrates a mechanism associated with activatable PARACEST MRI agents.

FIG. 2 illustrates a mechanism associated with deactivatable PARACEST MRI agents.

FIG. 3 illustrates a reaction of Yb-DO3A-anilide with NO to form Yb-DO3A-triazine.

FIG. 4 illustrates PARACEST spectra of Yb-DO3A-anilide and Yb-DO3A-triazine.

FIG. 5 illustrates a reaction of Yb(III)DO3A-oAA with NO to form a triazene product.

FIG. 6 illustrates the CEST spectrum of Yb(III)DO3A-oAA.

FIG. 7 illustrates a schematic of activatable PARACEST MRI agents that detect transglutaminase.

FIG. 8 illustrates PARACEST spectra of Yb-DO3A-pentylamine before and after the transglutaminase catalyzed reaction with Z-Gln-Gly.

FIG. 9 illustrates the conversion of DEVD-(Ln-DOTA) to amino-(Ln-DOTA) through cleavage by caspase-3.

FIG. 10 illustrates a schematic of activatable PARACEST MRI agents that detect protease biomarkers.

FIG. 11 illustrates a schematic of an activatable PARACEST MRI agent capable of detecting MMP-9.

FIG. 12 illustrates a schematic of activatable PARACEST MRI agents that detect kinase biomarkers.

FIG. 13 illustrates the reaction of TML(Yb-DO3A-oAA) and esterase enzyme.

FIG. 14 illustrates PARACEST spectra of TML(Yb-DO3A-oAA) before and after reaction with porcine liver esterase enzyme.

FIG. 15 illustrates the synthesis of DEVD-(Tm-DOTA) using FMOC chemistry.

FIG. 16 illustrates PARACEST spectra and MR parametric map of DEVD-(Tm-DOTA) amide before and after adding caspase-3.

FIG. 17 illustrates the correlation of concentration and PARACEST of DEVD-(Tm-DOTA) amide using modified Bloch equations.

FIG. 18 illustrates the effect of pH and temperature on PARACEST of DEVD-(Tm-DOTA) amide.

FIG. 19 illustrates MRI images of Yb(III)DO3A-oAA and a triazene product.

FIG. 20 illustrates the relationship between concentration and PARACEST effect.

FIG. 21 illustrates a triple-reporter contrast agent.

FIG. 22 illustrates a schematic of an activatable PARACEST agent that can detect Cathepsin B.

### DETAILED DESCRIPTION

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

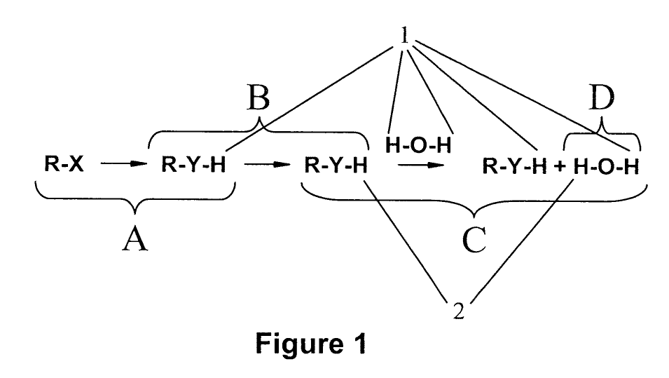

In one embodiment, a magnetic resonance imaging (MRI) contrast agent that may be detected via Chemical Exchange Saturation Transfer (CEST) is provided. The CEST effect relies on a chemical functional group on the contrast agent. The chemical functional group (e.g., an imine, amide, amine, hydroxyl, thiol, or phosphate group) exchanges hydrogen atoms with water, altering the MR response of water. As shown in FIG. 1, the CEST effect can be changed by an enzyme that catalyzes the (dis)appearance of the contrast agent chemical functional group. The CEST effect can also be changed by chemical reactions with other molecules (e.g., metabolic products) that also cause the (dis)appearance of the contrast agent chemical functional group. This “activation” or appearance of the CEST effect can be detected using non-invasive MRI.

“Activation” of the disappearance of the CEST effect can also be detected using MRI. This may be referred to as a “deactivatable” CEST agent. A deactivatable CEST agent works with a substantially constantly detectable CEST MRI agent also included as a control. Therefore, activatable CEST MRI agents that are designed to undergo reactions or catalysis with specific enzymes or metabolic products can be used to detect the presence of biomarkers.

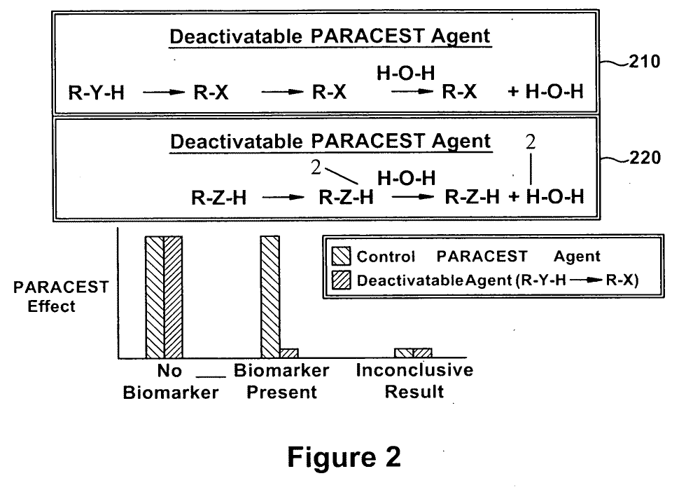

FIG. 2 provides a schematic of deactivatable PARACEST MRI agents. This type of PARACEST agent is compared to a control agent that exhibits a PARACEST effect. For example, if the deactivatable PARACEST agent 210 shows no PARACEST effect and yet the control agent 220 shows a PARACEST effect, and assuming that other conditions relevant for both agents are equal, then the biomarker that deactivates the agent is present. However, if both the deactivatable agent 210 and the control agent 220 show no PARACEST effect, effects other than presence of the biomarker may be responsible for reduced PARACEST effect of the deactivatable agent 210, so that the results are inconclusive. For example, poor in vivo pharmacokinetics of the deactivatable agent 210 and control agent 220 may result in poor PARACEST effects from both agents.

An activatable CEST MRI agent has a chemical functional group that exchanges hydrogens with water. This chemical functional group undergoes a change in chemical functionality due to enzyme catalysis or reaction with a metabolite. The change in chemical functionality causes a change in the chemical exchange rate and/or the MR frequency of the hydrogen exchange site. This change in chemical exchange rate and/or MR frequency is detectable using CEST MRI methods.

One embodiment concerns activatable PARACEST MRI agents that incorporate a lanthanide metal ion to shift the MR frequency of the chemical functional group to unique frequencies within the MR frequency spectrum. It will be appreciated by one skilled in the art that a paramagnetic lanthanide metal ion is not required in all embodiments.

Activatable CEST MRI agents mitigate issues associated with activatable relaxivity-based MRI agents. In one example, activatable CEST agents can be designed to be detected through different MR frequencies, which provides the opportunity to selectively detect multiple agents applied to the same study. Furthermore, activatable CEST agents can exhibit changes in MR frequencies following reaction with a biomarker (e.g., enzyme, metabolite), which provides a first sensitive method for detecting a biomarker. In addition, activatable CEST agents can exhibit changes in the range of 1-5000 sec⁻¹ in chemical exchange rates, which provides a second sensitive method for detecting reactions with enzymes or metabolites.

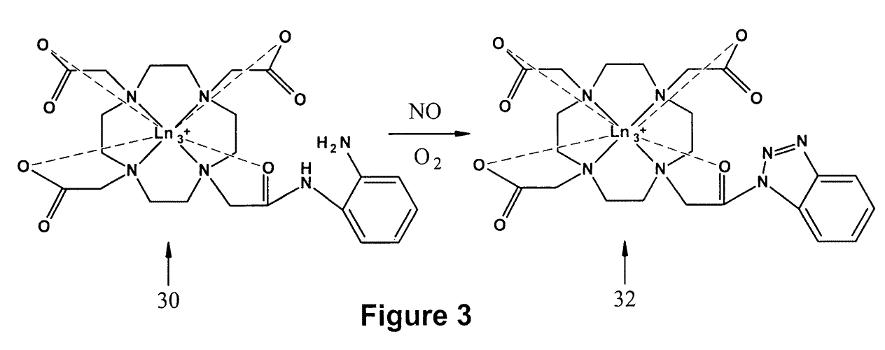

In one embodiment, as shown in FIG. 3, the activatable CEST MRI contrast agent may be Yb-DO3A-ortho-aminoanilide 30. FIG. 3 illustrates the reaction of Yb-DO3A-ortho-aminoanilide 30 with NO to form Yb-DO3A-triazine 32. The reactant 30 has a strong PARACEST effect, while the product 32 does not have a PARACEST effect.

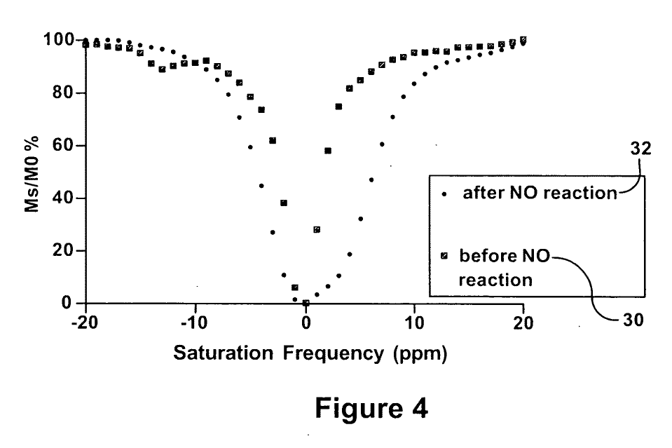

As shown in FIG. 4, CEST MR spectra of 30 and 32 in independent samples show a change in the CEST effect after reaction with nitric oxide. MR saturation at +10 ppm (amino resonance frequency) relative to the water resonance causes a decrease in MR water signal for a solution of Yb-DO3A-ortho-anilide 30. Similar MR saturation of a solution of Yb-DO3A-triazene 32 at +10 ppm shows no PARACEST effect. Direct MR saturation of the water at 0 ppm (the water resonance) also causes a decrease in water signal. Because direct water saturation is symmetric about 0 ppm, a comparison of water signal after saturation at +10 ppm vs −10 ppm is used to eliminate the effects of direct MR saturation of the water signal. Characterizing the product after reaction with nitric oxide verifies that the expected product is formed. Characterizing the change in CEST effect relative to concentration, temperature and pH demonstrates that this agent can be detected with good sensitivity under physiological conditions.

Thus, in one embodiment, an activatable PARACEST MRI contrast agent for detecting nitric oxide is provided. Nitric oxide (NO) is a versatile free radical molecule that is involved in physiological and pathological processes. NO can be detected using fluorescence imaging dyes, but this detection is very often limited to in vitro analyses due to problems with depth of penetration within in vivo tissues. Aromatic amines are known to specifically react with NO in the presence of oxygen to produce triazenes, causing a loss of the exchangeable protons.

In one embodiment, an activatable PARACEST MRI contrast agent exploits this mechanism to detect NO. The loss of exchangeable protons caused by a chemical reaction between NO and aromatic amines may be detected by a loss or “deactivation” of the PARACEST effect in MR images.

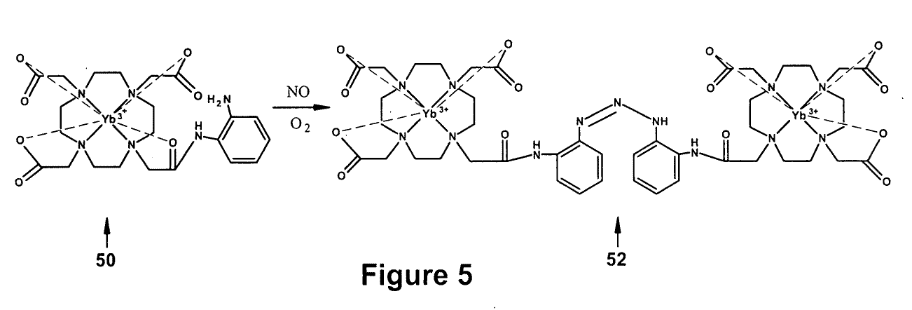

FIG. 5 illustrates an activatable PARACEST MRI contrast agent, Yb(III)DO3A-oAA 50, that has been designed and characterized to detect Nitric Oxide(NO). The agent 50 exhibits two CEST effects at −13 ppm and 10 ppm, corresponding to protons on amide and amine functional groups respectively. NO can effectively deactivate the CEST signal in a detectable range for MRI, so that the agent can be used for molecular imaging of NO.

As shown in FIG. 5, the reaction of 50 with NO in the presence of oxygen may convert aromatic amines to a triazene product 52. The loss of amine protons and the loss of the proximity of the amide proton to the lanthanide ion in 52 deactivates the PARACEST effect exhibited by 50. In one embodiment, a derivative of DOTA (1,4,7,10-tetraazacyclododecane-1,4,7,10-tetraacetic acid) was characterized with an ortho-aminoanilide motif. This product was used to chelate Yb(III) to form 50, which shifted the MR frequencies of the aromatic amide and amine to −13 and +10 ppm relative to the MR frequency of water. This product was also used to chelate other lanthanide ions. However, the Yb chelate demonstrated the strongest PARACEST effect due to its good compromise between T1 relaxation and ability to shift the MR frequency of the amide and amine protons.

The reaction conditions to form 52 were 40 mM of the contrast agent at pH 7.2 in one milliter of solution. 40 mg of NONOate was added, and O₂ was bubbled through the solution for 1 hour at 37° C., which produced an excess of NO and O₂. The pH of 7.2 allowed the PARACEST effect of both the amine and amide to be seen before the reaction.

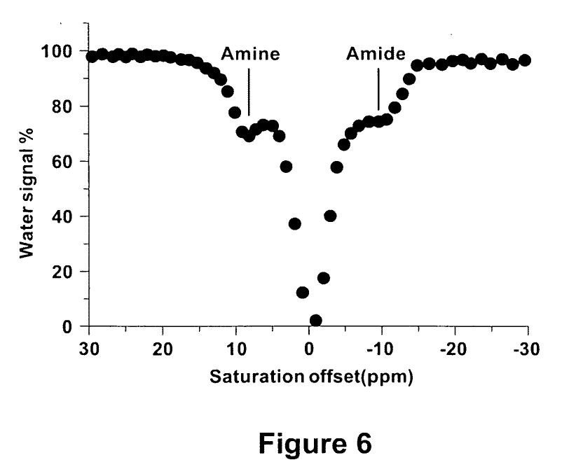

FIG. 6 shows the CEST spectrum of 40 mM of 50 at pH 7.0 and 37° C. The CEST spectrum was obtained on 600 MHz Varian Inova NMR spectrometer by a modified presaturation pulse sequence with saturation power of 523 Hz for 3 seconds applied in 1 ppm saturation offset increments from 30 ppm to −30 ppm. Two PARACEST peaks were observed at −13 ppm and +10 ppm, which is identical to the chemical shifts of exchangeable protons obtained from 1H NMR spectra. Direct saturation of the water MR signal was observed at 0 ppm.

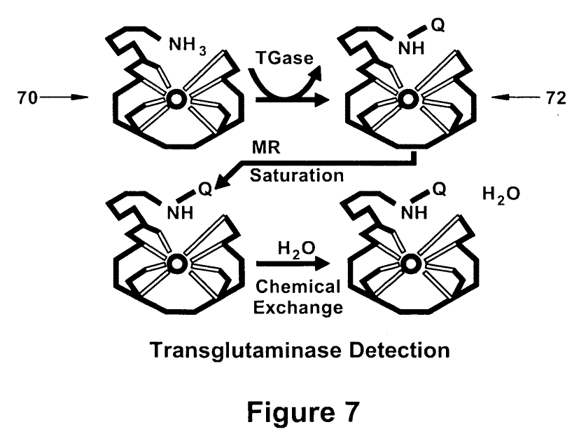

FIG. 7 provides a schematic of activatable PARACEST MRI agents that detect transglutaminase. In one embodiment, the activatable CEST MRI contrast agent may be Yb-DO3A-pentylamine 70. Reacting 70 with transglutaminase in the presence of a Z-Gln-Gly peptide that contains a glutamine residue produces the cross-linked peptide product 72. Comparision with FIG. 1 shows that X=aliphatic amine and Y=aliphatic amide of glutamine (Q=Glutamine).

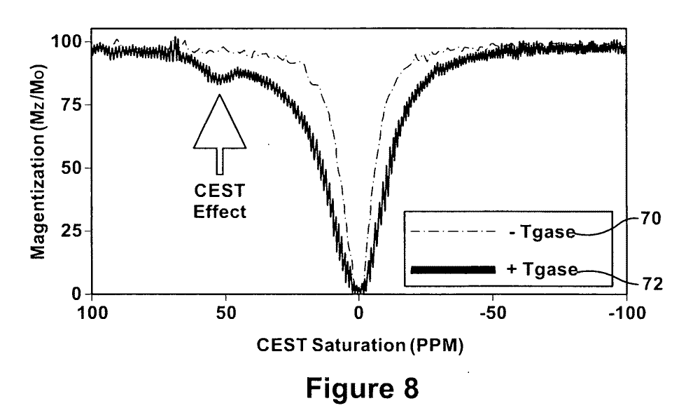

FIG. 8 provides a PARACTEST spectra of Yb-DO3A-pentylamine before 70 and after 72 the Transglutaminase-catalyzed reaction with Z-Gln-Gly. CEST MR spectra of 70 and 72 in independent samples show the appearance of the CEST effect after the reaction catalyzed by transglutaminase. MR saturation at +52 ppm relative to the water resonance causes a decrease in MR water signal for a solution of the Yb-DO3A-pentyl-(Z-Gln-Gly) product 72 formed via the Transglutaminase-catalyzed reaction (solid line). Similar MR saturation of a solution of Yb-DO3A-pentylamine 70 shows no PARACEST effect (dotted line). Direct MR saturation of the water at 0 ppm (the water resonance) causes a decrease in water signal for both samples. Because direct water saturation is symmetric about 0 ppm, a comparison of water signal after saturation at +52 ppm vs −52 ppm is used to eliminate the effects of direct MR saturation of the water signal.

An activatable CEST MRI contrast agent may be employed in different applications. By way of illustration, the agent may be used in diagnosing patients with disease states of biological processes that contain enzymatic or metabolic biomarkers and/or assessing the effect of therapies administered to these patients. The enzyme or metabolite may cause a change(s) in a chemical functional group of the MRI agent that results in a detectable change in the CEST effect. The agent is well-suited to patients or disease states that are assessed using non-invasive methods. Different applications of the agent are described below.

In one embodiment, activatable CEST MRI agents may be employed to assess metastasis, arthritis, and/or cell apoptosis by detecting protease enzymes. Protease enzymes degrade other proteins, and degradations of the extracellular matrices of proteins occur in many biological processes. For example, Matrix Metalloproteinases (MMPs) degrade proteins to clear away pathways for tumor cells to escape tumor tissues and metastasize to other tissues. MMPs also degrade proteins in cartilage to alleviate inflammation, which results in long-term loss of cartilage and the onset of osteoarthritis. Therefore, non-invasive detection of MMPs may facilitate detection of tumor metastasis, arthritis, and other diseases dependent on protein matrix degradations.

Protease enzymes also cleave other proteins to initiate metabolic pathways within cells. For example, caspases cleave inactive forms of other proteins (including other members of the caspase protease family), which activates these other proteins to perform their functions. This cleavage initiates the near-irreversible “death signaling cascade” that results on cell apoptosis. Therefore, caspase-3 is referred to as an “executioner” in the metabolic death cascade during cell apoptosis, and therefore serves as an early biomarker for evaluating apoptosis-promoting tumor therapies. Diseases involving aberrant apoptosis include cancer, hyperplasia, AIDS, allograft rejection, Alzheimer's disease, Parkinson's disease, autoimmunity (rheumatoid arthritis, type-I diabetes, lupus), restenosis, heart failure, stroke, inflammation, and trauma. Non-invasive detection of caspases may facilitate early detection of these diseases.

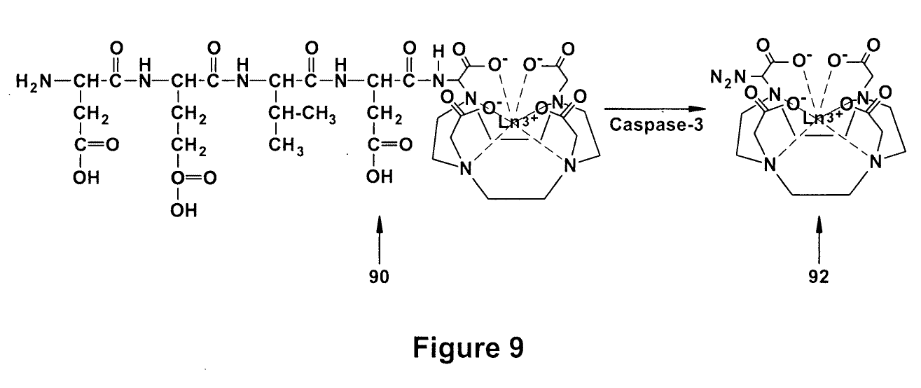

FIG. 9 illustrates an activatable PARACEST MRI contrast agent synthesized to measure apoptosis by detecting caspase-3. Among the identified substrates of caspase-3, DEVD (Asp-Glu-Val-Asp) is efficiently and selectively cleaved by caspase-3 and has been incorporated in fluorescence dyes for detecting caspase-3 (e.g., DEVD-AMC). In one embodiment, an activatable PARACEST MRI agent replaces AMC with DOTA (1,4,7,10-Tetraazacyclododecane-1,4,7,10-tetraacetic acid).

In one embodiment, the caspase-3 substrate DEVD (Asp-Glu-Val-Asp) was elongated using the amino group on one side arm of lanthanide ligand anchored on the polymer support. FIG. 9 illustrates the conversion of the DEVD-(Ln-DOTA) 90 to amino-(Ln-DOTA) 92 through cleavage by caspase-3. In one embodiment, an amide of DEVD-(Tm-DOTA) showed a PARACEST effect with MR saturation at −51 ppm. DEVD-(Tm-DOTA) amide was cleaved by caspase-3 exposing the free amine group, which showed PARACEST with saturation at +8 ppm. Accordingly, the enzymatic activity of caspase-3 can be detected by the change in PARACEST effect caused by this biotransformation.

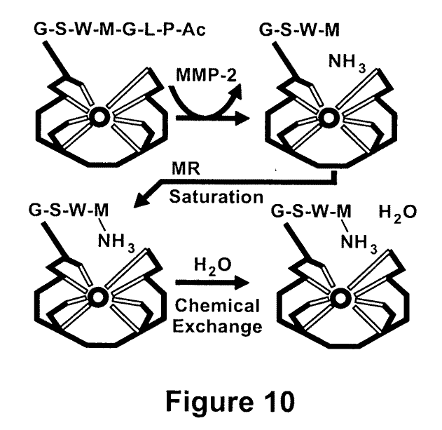

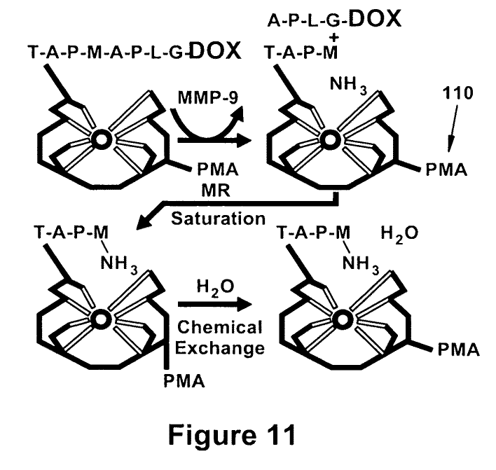

FIG. 10 illustrates the mechanism of detecting protease enzymes with an activatable PARACEST MRI agent. This mechanism constitutes a technology platform, whereby a range of peptidyl ligands can be used to detect different proteases. One embodiment detects MMP-2, a protease enzyme that is a biomarker for metastatic cancer. Comparison with FIG. 1 shows that X=full-length peptide before cleavage and Y=truncated peptide after cleavage. In another embodiment, as shown in FIG. 11, MMP-9 may be detected. As shown in FIG. 11, the contrast agent may be linked to a polymer 110.

In another embodiment, an activatable CEST MRI agent may be employed to assess cell signaling processes by detecting kinase enzymes. Kinase enzymes are responsible for a wide variety of cell signaling events in many biological processes. For example, HER2 is a tyrosine kinase cell receptor that is strongly linked to breast cancer metastases. When HER2 is stimulated through binding of an extracellular protein to its extracellular domain, the intracellular domain of HER2 can add a phosphate group to specific peptide sequences. This kinase event initiates a cascade of metabolic activity within the cell that eventually leads to cell metastasis.

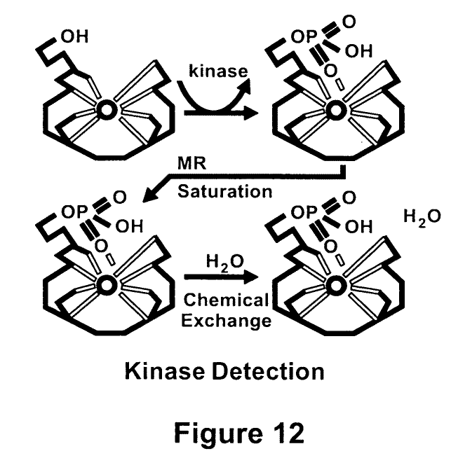

FIG. 12 illustrates the mechanism of detecting kinase enzymes with an activatable PARACEST MRI agent. Comparison with FIG. 1 shows that X=ligand before phosphorylation and Y=ligand after phosphorylation. The phosphorylated ligand may include a peptide or aliphatic linker that is targeted by a specific kinase. This mechanism also constitutes a technology platform, whereby a range of peptidyl ligands can be used to detect different proteases.

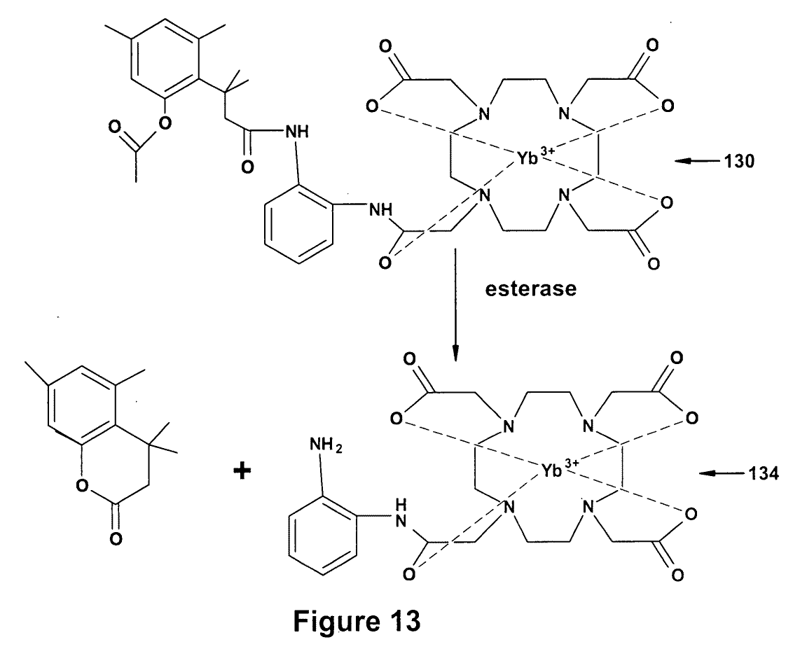

FIG. 13 illustrates an activatable CEST MRI agent to assess cell signaling processes by detecting esterase enzymes. As set forth above, PARACEST MRI contrast agents may detect enzyme activity by monitoring changes in the PARACEST effects from amine and/or amide groups after these groups undergo conversion to new chemical groups. This methodology can be extended to detect esterase enzymes by conjugating a PARACEST MRI contrast agent to a ‘trimethyl lock’ moiety. This moiety can be de-esterified by esterase enzymes, which triggers a self-immolative reaction that results in a product that exhibits two PARACEST effects. Because esterase enzymes are predominantly located within cells, such PARACEST MRI contrast agents may be used to track intracellular delivery.

Esterase enzymes are an attractive objective for molecular imaging because they are predominantly located within live cells, which can be used as a biomarker for intracellular delivery. Unfortunately, ester groups do not possess hydrogens and therefore can not produce a PARACEST effect. A ‘trimethyl lock’ moiety may undergo self-immolation following de-esterification, which converts an amide to an imine or amine. Therefore, conjugating this moiety to a PARACEST MRI contrast agent may modulate the PARACEST effect in response to esterase activity.

Yb(III)-1,4,7,10-tetraazacyclododecane-1,4,7,10-tetraacetic acid o-Aminoanilide (Yb-DO3A-oAA) was synthesized, and the product was confirmed by MS and NMR spectroscopy. The trimethyl lock {TML: 1-(1-dimethylcarboxyethyl)-2,4-methylphenylester} (Sigma Aldrich) was conjugated to the amine of Yb-DO3A-oAA, and this product was incubated with 3 units of porcine liver esterase enzyme (Calbiochem).

FIG. 13 illustrates a schematic of the reaction of TML(Yb-DO3A-oAA) 130 and esterase enzyme. PARACEST spectra of 25 mM of TML-(Yb-DO3A-oAA) were obtained before 130 and after 134 enzyme catalysis using a modified presaturation pulse sequence with a 600 MHz Varian NMR scanner.

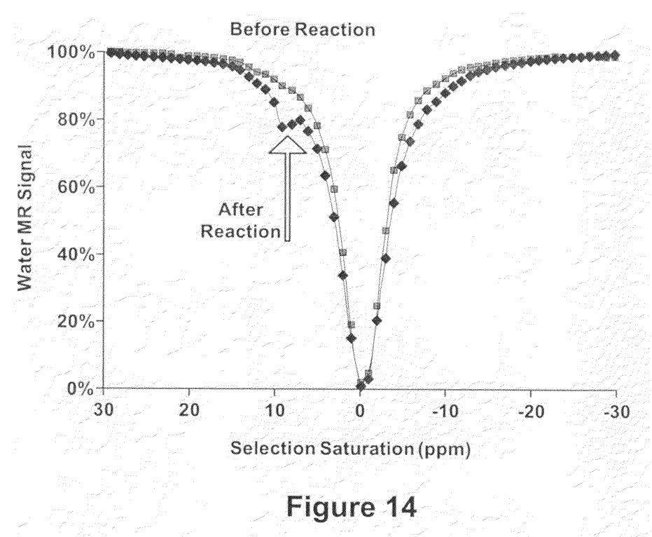

FIG. 14 illustrates PARACEST spectra of 25 nM of TML-(Yb-DO3A-oAA) before and after reaction with 3 units of porcine liver esterase enzyme. Spectra were acquired with a 4.2 μT preseaturation for 3 sec, at 37° C. TML-(Yb-DO3A-oAA) displayed no PARACEST effect prior to 130 the addition of the enzyme, and the product 134 of the reaction displayed a PARACEST effect at +10 ppm. This change in the PARACEST effect can be monitored using MR methods to detect esterase enzyme activities.

In another embodiment, an activatable CEST MRI agent may be employed to assess vascular remodeling and wound repair by detecting a cross-linking enzyme. Transglutaminase is responsible for cross-linking proteins in the extracellular matrix, which is involved in wound healing, stabilizing blood vessels after vascular remodeling and angiogenesis, and other biological processes. Transglutaminase links a primary amine group to a terminal amide. This reaction may be performed using lysine and glutamine side chains, but other aliphatic amines and aliphatic terminal amides may also be processed by transglutaminase. The mechanism of detecting transglutaminase with an activatable PARACEST MRI agent is shown in FIG. 7. This mechanism demonstrates that activatable CEST MRI agents can detect other enzyme-mediated reactions that do not cleave the ligand of the contrast agent but rather attach new substituents to the contrast agent.

In another embodiment, an activatable CEST MRI agent may be employed to assess tumor angiogenesis by detecting a metabolite. Nitric Oxide Synthase enzymes (NOS) are involved in several diseases and biological processes, including cell apoptosis, vascular inflammation, and atherosclerosis. Nitric oxide is a metabolic product of NOS. Because nitric oxide is rarely produced within biological systems without the presence of NOS, and because nitric oxide has a short lifespan, nitric oxide is a spatial and temporal indicator of NOS. Production of an amount of nitric oxide molecules per molecule of NOS causes a large relative abundance of nitric oxide that improves detection of this biomarker.

As shown in FIG. 3, nitric oxide reacts with oxygen and an analide to produce a triazene. This reaction mechanism is exploited by a commercially available fluorescence dye that detects nitric oxide. By conjugating an aminoanilide ligand to a core structure, it can be demonstrated that this MRI contrast agent changes its CEST effect after reacting with nitric oxide. This mechanism demonstrates that activatable CEST MRI agents can detect other molecular biomarkers besides enzymes.

The reaction conditions to form 32 were 1 mM of the contrast agent 30 in 5 mL of distilled water, and with 40 mg of NONOate to produce an excess of NO. The reaction was carried out at 37° C. for one hour. As illustrated in FIG. 4, the lower pH after reaction in unbuffered solution of approximately 5.7-6.3 only allowed the CEST effect from the amine to be observed.

In one embodiment, enzymatic catalysis may be exploited to change the chemical structure of a high concentration of PARACEST agents and cause a detectable change in the PARACEST effect. The high catalytic activity may facilitate indirectly detecting a relatively low concentration of the enzyme. By exploiting enzyme activity instead of the presence of the enzyme, as little as about 3.4 nM of active enzyme may be detected within 20 minutes after applying the PARACEST agent, and about 5 nM of active enzyme may be detected within 10 minutes after applying the PARACEST agent. For example, enzymatic conversion of an amide to an amine will accelerate the chemical exchange rate between the agent and water from ˜300 sec⁻¹ to ˜3000 sec⁻¹. Additionally, the MR chemical shift frequency of the amide and amine will be significantly different, especially if these functional groups are proximal to a paramagnetic lanthanide ion. The chemical shift change may be advantageous for detection because MR methods are sensitive to changes in MR frequencies.

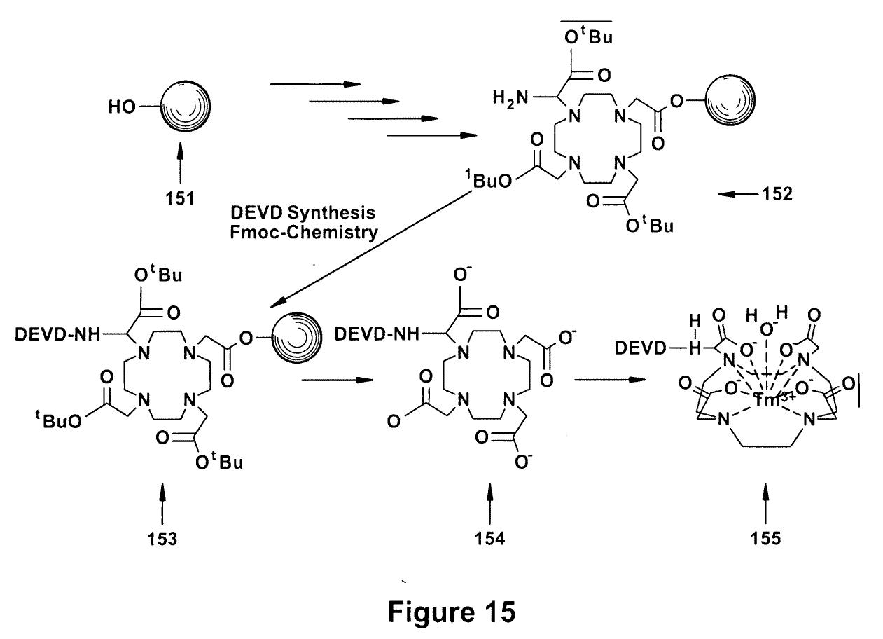

FIG. 15 illustrates one example of the synthesis of DEVD-(Tm-DOTA) using Fmoc chemistry. To synthesize DEVD-DOTA amide 154, a polymer support pre-loaded with a DOTA derivative 152 was developed. Standard Fmoc solid phase peptide synthesis methods were then used to “grow” the DEVD peptide chain onto the amino group of 152. Following the synthesis, the acquired compound 154 was characterized with MALDI-MASS (m/z 885.80 [M+H]). Thulium was chelated with 154 to prepare the final compound 155 (m/z 1088.74 [M+Na]⁺). In one embodiment, final compound 155 was used to detect the activity of caspase-3.

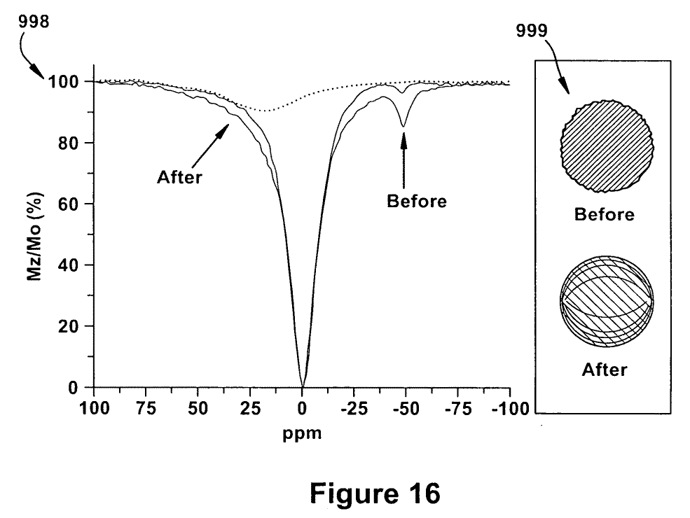

FIG. 16 illustrates PARACEST spectra 998 and MR parametric map 999 of DEVD-(Tm-DOTA) amide 155 before and after adding caspase-3. PARACEST spectra were acquired at 37° C. and pH 7.4 with a continuous wave saturation pulse applied at 31 μT for 4 seconds. The deconvoluted PARACEST spectrum of the product after reaction, showing a PARACEST effect at +8 ppm, is also shown. MR images were acquired at 37° C. and pH 7.4 with a Bruker Biopsin 9.4 T MR scanner. A MSME T1 method was used with TR/TE=1623/10.9 ms and a train of Gaussian-shaped saturation pulses applied at 25 μT for 1.106 s, and with saturation offsets at −51 ppm and +51 ppm. The parametric map was obtained by subtracting the MR image with a saturation offset at −51 ppm from the MR image with saturation offset at +51 ppm. The magnitude of the scale of the original MR images was used as the scale for this parametric map, to properly represent the difference in MRI contrast obtained with different saturation offsets. The PARACEST spectrum of 155 (25 mM, pH 7.4) was recorded by applying selective saturation in 1 ppm increments from +100 ppm to −100 ppm. A PARACEST effect was detected at −51 ppm, which was assigned to the amide most proximal to the lanthanide ion in 155, based on identical results obtained from a similar compound, Tm3⁺-DOTAMGly. After 48 nM of caspase-3 was added and the mixture was incubated at 37° C. and pH 7.4 for 1 hour, the PARACEST effect at −51 ppm was decreased and an asymmetrical shape in the PARACEST spectrum was observed near water. This asymmetry was analyzed by deconvolution to show a PARACEST effect at +8 ppm. Considering that the PARACEST spectrum of 152 also shows an identical PARACEST peak at +8 ppm this effect further confirms that caspase-3 has converted the DOTA-amide of 155 to the DOTA-amine of 152.

FIG. 16 illustrates an MR image acquired with selective saturation at −51 ppm was acquired with 155 before and after reaction with caspase-3. An MR image with selective saturation at +51 was also acquired as a control to account for direct saturation of water. The difference between these images showed approximately a 14.5% decrease in water MR signal before the enzymatic reaction due to the PARACEST effect, and no significant change in water MR signal after reaction.

To determine the sensitivity of detecting 155 under physiological conditions, the PARACEST effect of the agent was correlated with concentrations using modified Bloch equations. A modified Bloch equation for two proton pools undergoing exchange was used to describe the relationship of the PARACEST effect and concentration of 155 (equation 1).

$$\frac{Ms}{M 0} = \frac{1}{1 + \frac{{n}_{CA} [ C A ] {T}_{1 sat}}{{n}_{H 2 O} [ H 2 O ] {τ}_{M}}} \qquad ( 1 )$$

Mₛ: MR signal of water proton pool during selective saturation of the contrast agent proton pool
M₀: MR signal of water proton pool without selective saturation
n_{CA}: number of exchangeable protons of the contrast agent proton pool
n_{H2O}: number of exchangeable protons of the water proton pool (2)
[CA]: concentration of contrast agent
[H₂O]: concentration of water (˜55 M)
T₁ₛₐₜ: T₁ relaxation time constant of the water proton pool during selective saturation of the contrast agent proton pool
T_{M}: average lifetime of the proton on the contrast agent

1/T₁ₛₐₜ was found to be linearly related to contrast agent concentration by using a T₁ inversion recovery method with selective saturation at the amide or amine chemical shifts. By substituting T₁ₛₐₜ with a linear relationship based on [CA], the modified Bloch equation can be further simplified (equation 2), where m and b represent the slope and intercept of the linear relationship between T₁ and [CA]. This equation was exploited to determine the sensitivity of detecting contrast agent 155.

$$\frac{1}{[ C A ]} = \frac{1}{( \frac{{M}_{0}}{{M}_{z}} - 1 )} [ \frac{{n}_{CA}}{{bn}_{H 2 O} {τ}_{M}} ] - \frac{m}{b} \qquad ( 2 )$$

After validating a linear relationship between concentration and T₁ relaxation under selective saturation conditions, and after confirming that the selective saturation pulse was sufficiently long to achieve steady-state conditions, this approach was further modified to obtain a linear relationship that correlates concentration to the PARACEST effect.

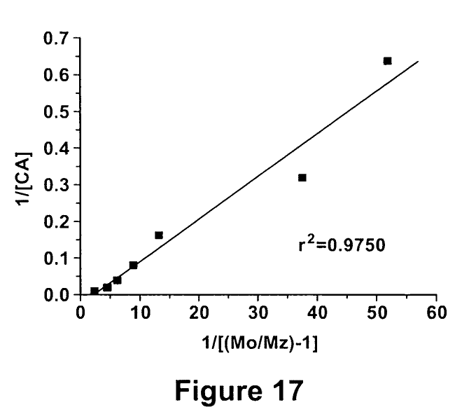

FIG. 17 illustrates the correlation of concentration and PARACEST of DEVD-(Tm-DOTA) amide using Bloch equations. PARACEST was measured at 37° C. and pH 7.4, using a continuous wave saturation pulse applied at −51 ppm and +51 ppm at 31 μT for 4 seconds. These results indicate that 0.90 mM of the agent can be detected by saturating the amide MR frequency to generate a 1% change in water MR signal.

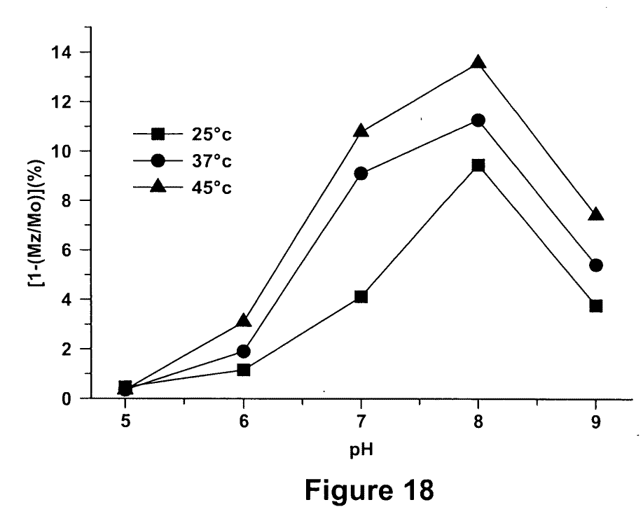

pH can also influence the PARACEST effect because proton chemical exchange between water and amides is catalyzed by hydroxide ions. FIG. 18 illustrates the effect of pH and temperature on PARACEST of 25 mM DEVD-(Tm-DOTA) amide. PARACEST was measured using a continuous wave saturation pulse applied at −51 ppm and +51 ppm at 31 μT for 4 seconds. FIG. 18 illustrates that the amide proton showed increasingly greater PARACEST with increasing pH, reaching the greatest effect at near pH 8. The proton chemical exchange rate between an amide and water is approximately 300 Hz. 300 Hz is relatively slow on the MR time scale, which is characterized by the chemical shift difference between the amide and water (30,600 Hz at 14.1 T). Therefore, an increasing hydroxide ion concentration accelerates this rate to improve the PARACEST effect. pH also influenced the PARACEST effect of the amine. The amine protons showed increasingly greater PARACEST with decreasing pH, reaching the greatest effect at pH 5. The proton chemical exchange rate between an amine and water is approximately 3000-5000 Hz, which is relatively fast on the MR time scale (compared to the chemical shift difference between the amine and water (4,800 Hz at 14.1 T), and decreasing hydroxide ion concentration decelerates this rate to improve the PARACEST effect. This further confirmed that these two PARACEST effects do not arise from metal-bound water, which does not exhibit a pH dependent PARACEST effect.

MRI contrast agents undergo a permanent structural change through enzymatic catalysis that causes a change in contrast within relaxation-weighted MR images. The absolute sensitivity of relaxivity-based MR agents has been shown to be 1-2 orders of magnitude better than the sensitivities of PARACEST agents. However, the ability to selectively detect PARACEST agents may provide additional advantages. For example, an enzymatically inert PARACEST agent with a unique saturation frequency may be directly linked to 155 to account for variances in concentration. This facilitates validating caspase-3 activity detection during in vivo biomedical applications.

DEVD-(Tm-DOTA) amide 155 shows PARACEST with good sensitivity at physiological pH and temperature, indicating that this MRI contrast agent can be used for in vivo molecular imaging. The detection of catalytic activity of caspase-3, rather than the presence of caspase-3, can facilitate molecular imaging. A relatively low concentration of enzymes with rapid activity can quickly convert a high concentration of MRI contrast agents for detection using PARACEST MR methods. Caspase-3 is constitutively expressed as an inactive proenzyme, so that detecting enzyme activity avoids detection of the inactive form. Specificities for different substrates are relatively good for different members of the caspase enzyme family, so that detecting enzyme activity can exploit substrate specificity. Finally, a variety of enzyme biomarkers can catalyze the conversion of amines, amides, and other functional groups that exchange protons with water. Therefore, in different embodiments, a “smart” PARACEST MRI contrast agent may have broad applicability for assessing enzyme biomarkers in biological processes and disease pathologies.

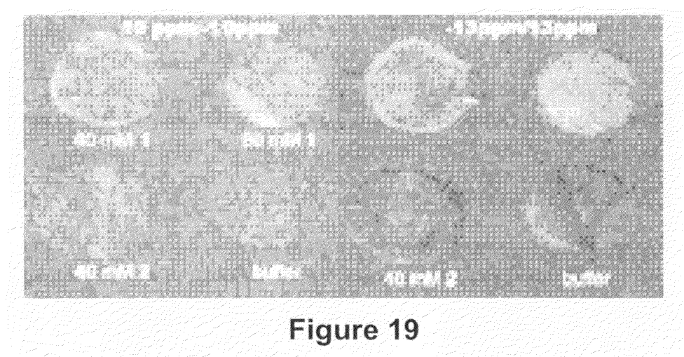

To illustrate that NO can effectively “deactivate” the PARACEST effect, 40 mM of 50 was combined with an excess amount of NONOate at pH 7.0 and 37° C. for 1 hour, to simulate the production of NO under physiological conditions. Complete conversion of the aromatic amine and amide to a triazene was confirmed by mass spectrometry (MALDI m/z1339). FIG. 19 illustrates MR images of this reaction mixture. Unreacted images of 50, and a water phantom (10 mM PBS buffer) were acquired with selective saturation at −13 ppm, 13 ppm, 10 ppm and −10 ppm. T1-weighted MRI images of phantoms containing PBS buffer, 80 mM 50, 40 mM 50, and 40 mM 52 (product of 40 mM 50 with NO) were acquired. Images were collected at room temperature on Bruker Biopsin 9.4 T small animal scanner. A MSME T1 method with TR/TE=3282/10.9 ms was used with 1000 Gauss shaped saturation pulses, saturation power of 12.3 uT, saturation delay of 2.25 s, and saturation offsets at −13 ppm, 13 ppm, 10 ppm and −10 ppm. The images were obtained by subtracting the images with saturation offset at 13 ppm by the images with saturation offset at −13 ppm (right) and the images with −10 ppm saturation offset by images with saturation offset at 10 ppm (left) to account for direct saturation of water. The image grayscale was inverted to show negative pixel values as bright. The magnitude of the grayscale of the original MR images was used as the scale for this displayed difference image, to properly represent the difference in MRI contrast obtained by comparing results with different saturation offsets. These results demonstrated that reaction with NO effectively deactivates the PARACEST effect of 50, so that this activatable PARACEST MRI contrast agent can be used to detect NO.

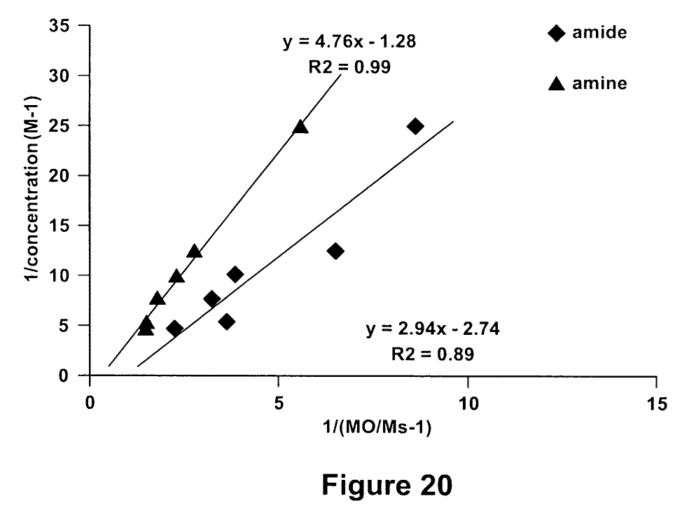

To illustrate the sensitivity of detecting 50, the PARACEST effect of the agent was correlated with concentration using modified Bloch equations. After validating a linear relationship between concentration and T1 relaxation under each saturation condition, the approach was further modified to obtain a linear relationship that correlates concentration to the PARACEST effect, as shown in FIG. 20. If a 1% change in MRI signal is considered to be the minimum threshold for detecting the PARACEST agent, these results indicate that about 2.1 mM and about 3.5 mM of agent can be detected by saturating amine and amide MR frequencies, respectively. The presence of two PARACEST frequencies from the same MRI contrast agent is a unique property of 50, which provides the opportunity for simultaneous saturation at both MR frequencies that may reduce this minimum detection threshold to about 1.3 mM.

A “deactivatable” PARACEST MRI contrast agent requires an “unactivatable” agent to serve as a control, in order to confirm that an absence of a PARACEST effect is due to reaction of 50 with NO. Selective detection of two PARACEST agents can be accomplished during the same MRI scan session, which facilitates the inclusion of this “unactivatable” agent within the MRI protocol.

In summary, 50 represents an activatable molecular imaging agent that can detect chemical and biochemical environments through modulation of the PARACEST effect as detected by MRI. This activatable PARACEST MRI contrast agent can be selectively detected, and detected with good sensitivity at high magnetic fields, which overcomes technological hurdles with relaxivity-based MRI agents. The concentration of the contrast agent can be quantified, and the effects of temperature and pH can be considered. Because a variety of amides and amines are modified by biochemical events in physiological processes, this initial demonstration represents a platform technology for designing new activatable molecular imaging agents to address diverse biomedical applications.

The selective saturation of PARACEST MRI agents allows for the use of multiple agents with unique PARACEST frequencies for selective detection. In one embodiment, an autophagin-1-detecting PARACEST agent may be combined with a caspase-3-detecting PARACEST agent to simultaneously monitor apoptosis and authophagy in response to rapamycin treatment. Rapamycin has been reported to induce apoptosis, autophagy, and vascular collapse in various in vitro and in vivo cancer models.

Selective detection via PARACEST also provides opportunities to include additional MRI contrast agents to quantify concentrations of the agents within intracellular and extracellular tissue volumes. In one embodiment, an enzyme-unresponsive agent may be linked to an enzyme-detecting agent to create a multi-reporter agent. The enzyme-unresponsive agent can be used to monitor extracellular and intracellular concentrations of the multireporter agent. In other embodiments, a DCE MRI agent (used to measure the dynamic uptake of a standard relaxivity-based MRI contrast agent in the extracellular volume of tumor tissues) may be combined with an enzyme-responsive PARACEST agent to simultaneously monitor vascular collapse, apoptosis, and autophagy in response to rapamycin.

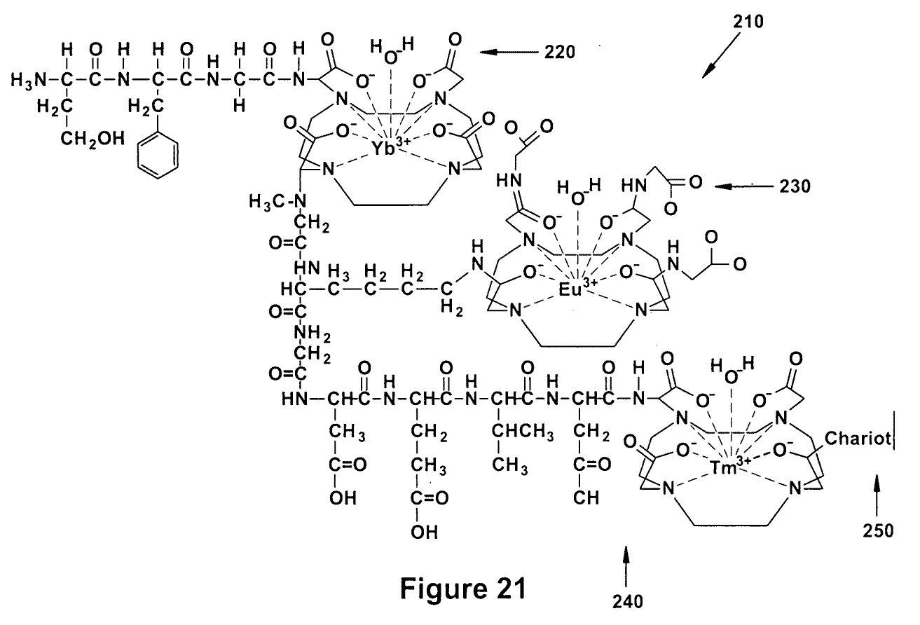

In another embodiment, a triple-reporter PARACEST MRI contrast agent may be used that detects caspase-3 and autophagin-1 enzymatic activity. FIG. 21 illustrates a triple-reporter PARACEST agent 210 that may simultaneously monitor caspase-3 activity (with PARACEST contrast agent 220) and autophagin-1 activity (with PARACEST contrast agent 230) relative to an enzyme-unresponsive PARACEST agent 240. All three PARACEST agents may be covalently linked to eliminate potential complications from differential pharmacokinetics, and to reduce monomer concentrations of the final formulation. The triple-reporter PARACEST agent 210 may also be covalently coupled to Chariot 250, a non-cytotoxic cell-penetrating peptide, to facilitate intracellular delivery.

PARAmagnetic MRI contrast agents can also be used to simultaneously report on the delivery of the drug to the tissue of interest and the release of the drug from the delivery system within the tissue. PARACEST agents may be conjugated to a hydroxypropylmethyacrylate (HPMA) polymeric drug delivery nanocarrier, dendrimers that detect tumor pH, liposomes that carry PC4 drug payloads for antitumor photodynamic therapy, and polylysine gene delivery nanocarriers.

A hydroxylaminepropylmethacrylate polymer (HAPMA) has been synthesized, and the PARACEST agent has been derivatized to contain an α-ketocarboxylate ligand. Because hydroxylamine and α-ketocarboxylate moieties efficiently couple and are unreactive with other functional groups, this bioorthogonal approach allows conjugation of the PARACEST agent to the polymer without the complication of side reactions. This bioorthogonal synthesis method provides a method to “click” MRI contrast agents onto nanoparticles for a variety of applications.

pH-responsive MRI contrast agents have been conjugated to dendrimers to create an agent that has 1000-fold improvement in detection sensitivity and 5-fold improvement in sensitivity for detecting small pH gradients between different tissues. The dendrimers have been biotinylated so that a biotin-avidin system can be used to target the nanoparticles to the liver. This nanoscale MRI contrast agent can be applied to detect hepatoccellular carcinoma by measuring differences in pH between tumors and normal liver tissues

The PARACEST agents have also been incorporated into liposomes, by conjugating the agents to the surface of a liposome, and by entrapping different PARACEST agents within the liposome core. The conjugated PARACEST agents are used to report on the pharmacokinetic delivery of the liposomal nanoparticle, while the entrapped PARACEST agents report on the degradation of the nanoparticle.

In addition, a cell-penetrating peptide may be labeled with a SPECT chelator. Synthesis of the peptidyl chelator may be coupled to a pegylated polylysine gene delivery nanocarrier. After chelating In-111, the nanocarrier may be used to track biodistributions of the nanocarrier.

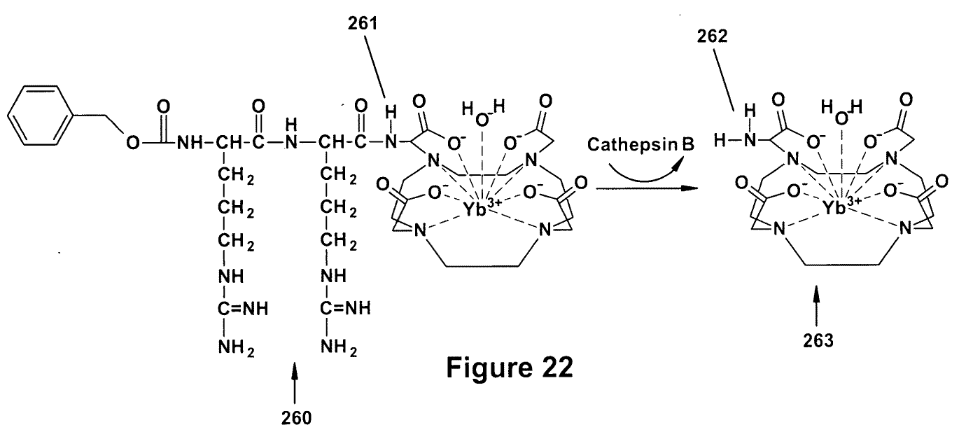

Further, FIG. 22 illustrates the cleavage of the peptidyl ligand of CBZ-Arg-Arg-(Yb-DOTA) 260 by Cathespsin B. Cathepsin B is an exopeptidase enzyme that is responsible for activating other enzymes that clear the extracellular matrix to provide avenues for tumor cell metastasis in breast tumors. Accordingly, an enzyme-responsive PARACEST MRI contrast agent 260 may be provided that contains a peptidyl ligand that can be cleaved by Cathepsin B to cause an amide functional group 261 on the agent to be converted to an amine 262 to change the agent's 263 PARACEST effect.

Table 1, as set forth below, describes a set of proteases that that can be detected with the enzyme-responsive MRI contrast agents. The proteases in this table may be referred to as “Protease Set A.” Thus, when the term Protease Set A appears in the claims Applicants intend to refer to this set of proteases.

TABLE 1

| Peptidase | Class | Species |
|---|---|---|
| C01.007: actinidain | Cysteine Protease | Actinidia deliciosa (kiwi), |
| | | Freesia reflacta (plant) |
| C01.081: papain homologue (Dictyostelium- | Cysteine Protease | amoeba - slime mold |
| type) | | |
| M04.006: Msp peptidase (Legionella sp.) | Metallo Protease | bacteria |
| C25.001: gingipain R | Cysteine Protease | bacteria |
| C25.002: gingipain K | Cysteine Protease | bacteria |
| M20.008: carboxypeptidase Ss1 | Metallo Protease | bacteria |
| C11.001: clostripain | Cysteine Protease | bacteria |
| C47.001: staphopain A | Cysteine Protease | bacteria |
| C47.002: staphopain B | Cysteine Protease | bacteria |
| C47.003: ecp g.p. (Staphylococcus | Cysteine Protease | bacteria |
| epidermidis) | | |
| C54.001: ATG4 peptidase (Saccharomyces | Cysteine Protease | bacteria |
| cerevisiae) | | |
| C56.001: PfpI peptidase | Cysteine Protease | bacteria |
| M01.002: lysyl aminopeptidase (bacteria) | Metallo Protease | bacteria |
| M01.009: aminopeptidase N (actinomycete- | Metallo Protease | bacteria |
| type) | | |
| M01.020: tricorn interacting factor F2 | Metallo Protease | bacteria |
| (Thermoplasma sp.) | | |
| M01.021: tricorn interacting factor F3 | Metallo Protease | bacteria |
| (Thermoplasma sp.) | | |
| M13.005: oligopeptidase O3 | Metallo Protease | bacteria |
| M18.001: aminopeptidase I | Metallo Protease | bacteria |
| M28.001: aminopeptidase Y | Metallo Protease | bacteria |
| M28.003: aminopeptidase S | Metallo Protease | bacteria |
| M29.001: aminopeptidase T | Metallo Protease | bacteria |
| M29.004: PepS aminopeptidase | Metallo Protease | bacteria |
| M42.001: glutamyl aminopeptidase | Metallo Protease | bacteria |
| (bacterium) | | |
| M42.002: bacillus aminopeptidase I | Metallo Protease | bacteria |
| (Geobacillus/Bacillus stearothermophilus) | | |
| M61.001: glycyl aminopeptidase | Metallo Protease | bacteria |
| M75.001: imelysin | Metallo Protease | bacteria |
| M9A.005: clostridial aminopeptidase | Metallo Protease | bacteria |
| S01.101: trypsin (Streptomyces sp.) | Serine Protease | bacteria |
| S01.102: trypsin (Streptomyces erythreaus) | Serine Protease | bacteria |
| S01.262: streptogrisin B | Serine Protease | bacteria |
| S01.267: streptogrisin E | Serine Protease | bacteria |
| S01.268: alpha-lytic endopeptidase | Serine Protease | bacteria |
| S01.269: glutamyl peptidase I | Serine Protease | bacteria |
| S08.001: subtilisin Carlsberg | Serine Protease | bacteria |
| S08.007: thermitase | Serine Protease | bacteria |
| S08.008: Mername-AA053 peptidase | Serine Protease | bacteria |
| S08.009: subtilisin Ak1 | Serine Protease | bacteria |
| S08.017: bacillopeptidase F | Serine Protease | bacteria |
| S08.019: lactocepin I | Serine Protease | bacteria |
| S08.024: trepolisin | Serine Protease | bacteria |
| S08.051: aqualysin 1 | Serine Protease | bacteria |
| S08.053: oryzin | Serine Protease | bacteria |
| S08.056: cuticle-degrading peptidase | Serine Protease | bacteria |
| S08.079: PrcA peptidase | Serine Protease | bacteria |
| S08.091: tripeptidyl-peptidase S | Serine Protease | bacteria |
| S08.101: halolysin 1 | Serine Protease | bacteria |
| S08.102: halolysin R4 | Serine Protease | bacteria |
| S08.110: StmPr1 peptidase | Serine Protease | bacteria |
| (Stenotrophomonas-type) | | |
| S08.116: lactocepin III | Serine Protease | bacteria |
| S09.005: dipeptidyl aminopeptidase A | Serine Protease | bacteria |
| S09.008: dipeptidyl peptidase IV | Serine Protease | bacteria |
| (Aspergillus-type) | | |
| S09.010: oligopeptidase B | Serine Protease | bacteria |
| S14.001: peptidase Clp (type 1) | Serine Protease | bacteria |
| S15.001: Xaa-Pro dipeptidyl-peptidase | Serine Protease | bacteria |
| S16.001: Lon-A peptidase | Serine Protease | bacteria |
| S33.002: tripeptidyl-peptidase A | Serine Protease | bacteria |
| (Streptomyces sp.) | | |
| S33.006: tripeptidyl-peptidase B | Serine Protease | bacteria |
| S37.001: PS-10 peptidase | Serine Protease | bacteria |
| S46.001: dipeptidyl-peptidase 7 | Serine Protease | bacteria |
| S49.001: signal peptide peptidase A | Serine Protease | bacteria |
| S51.001: dipeptidase E | Serine Protease | bacteria |
| S58.001: aminopeptidase DmpA | Serine Protease | bacteria |
| S9G.064: archealysin | Serine Protease | bacteria |
| T01.002: archaean proteasome, beta | Threonine Protease | bacteria |
| component | | |
| T01.005: bacterial proteasome, beta | Threonine Protease | bacteria |
| component | | |
| T01.006: HslV component of HslUV | Threonine Protease | bacteria |
| peptidase | | |
| XP01-001: tricorn peptidase complex | compound Protease | bacteria |
| S01.090: hypodermin B | Serine Protease | cattle grub |
| S01.111: hypodermin A | Serine Protease | cattle grub |
| M01.016: aminopeptidase Ey | Metallo Protease | chicken, ostrich |
| C01.005: stem bromelain | Cysteine Protease | comosus (pineapple) |
| C01.026: ananain | Cysteine Protease | comosus (pineapple) |
| C01.027: comosain | Cysteine Protease | comosus (pineapple) |
| C01.028: fruit bromelain | Cysteine Protease | comosus (pineapple), corn |
| S01.001: chymotrypsin A (cattle-type) | Serine Protease | cow |
| S01.142: duodenase | Serine Protease | cow, chicken |
| C01.019: CC-I peptidase (Carica sp.) | Cysteine Protease | cundinamarcensis (papaya) |
| C01.020: CC-III peptidase (Carica | Cysteine Protease | cundinamarcensis (papaya) |
| candamarcensis) | | |
| C01.073: peptidase 1 (mite) | Cysteine Protease | dust mite |
| S01.031: peptidase 9 (Dermatophagoides- | Serine Protease | dust mite |
| type) | | |
| S01.187: peptidase 6 (Dermatophagoides | Serine Protease | dust mite |
| sp.) | | |
| S01.234: peptidase 3 (Dermatophagoides- | Serine Protease | dust mite |
| type) | | |
| XT01-001: 20 S proteasome peptidase | compound Protease | eukaryote |
| complex (eukaryote) | | |
| XT01-002: 26 S proteasome peptidase | compound Protease | eukaryote |
| complex (eukaryote) | | |
| M12.001: astacin | Metallo Protease | european crayfish |
| C01.006: ficain | Cysteine Protease | Ficus glabrata (wild fig) |
| S9G.065: fish muscle prokallikrein | Serine Protease | fish |
| C01.033: cathepsin L-iike peptidase | Cysteine Protease | Flatworm |
| (Fasciola sp.) | | |
| S01.126: Mername-AA135 trypsin | Serine Protease | frog, fish |
| S01.240: oviductin | Serine Protease | frog, toad |
| M01.006: Ape2 aminopeptidase | Metallo Protease | fungus |
| M35.002: deuterolysin | Metallo Protease | fungus |
| S09.006: dipeptidyl aminopeptidase B | Serine Protease | fungus |
| (fungus) | | |
| S09.012: dipeptidyl-peptidase V | Serine Protease | fungus |
| S01.219: coagulation factor C (horseshoe | Serine Protease | horseshoe crab |
| crab), activated | | |
| S01.220: coagulation factor B (Limulus, | Serine Protease | horseshoe crab |
| {Tachypleus}), activated | | |
| S01.221: clotting enzyme (Tachypleus) | Serine Protease | horseshoe crab |
| S01.222: coagulation factor G (Tachypleus), | Serine Protease | horseshoe crab |
| activated | | |
| S09.007: fibroblast activation protein alpha | Serine Protease | Human |
| subunit | | |
| S01.154: pancreatic endopeptidase E | Serine Protease | Human |
| M17.001: leucyl aminopeptidase (animal) | Metallo Protease | Human |
| M49.001: dipeptidyl-peptidase III | Metallo Protease | Human |
| S60.001: lactoferrin | Serine Protease | Human |
| S01.218: protein C (activated) | Serine Protease | Human |
| C14.001: caspase-1 | Cysteine Protease | Human |
| C14.003: caspase-3 | Cysteine Protease | Human |
| T02.001: glycosylasparaginase precursor | Threonine Protease | Human |
| S01.300: stratum corneum chymotryptic | Serine Protease | Human |
| enzyme | | |
| C19.001: ubiquitin-specific peptidase 5 | Cysteine Protease | Human |
| S01.133: cathepsin G | Serine Protease | Human |
| S01.213: coagulation factor XIa | Serine Protease | Human |
| S01.174: mesotrypsin | Serine Protease | Human |
| C13.004: legumain (chordate) | Cysteine Protease | Human |
| M01.004: leukotriene A4 hydrolase | Metallo Protease | Human |
| M01.001: aminopeptidase N | Metallo Protease | Human |
| S01.217: thrombin | Serine Protease | Human |
| S01.015: tryptase beta | Serine Protease | Human |
| S01.216: coagulation factor Xa | Serine Protease | Human |
| S01.233: plasmin | Serine Protease | Human |
| M01.010: cytosol alanyl aminopeptidase | Metallo Protease | Human |
| M01.011: cystinyl aminopeptidase | Metallo Protease | Human |
| M01.014: aminopeptidase B | Metallo Protease | Human |
| S01.151: trypsin 1 | Serine Protease | Human |
| S01.152: chymotrypsin B | Serine Protease | Human |
| S01.157: chymotrypsin C | Serine Protease | Human |
| S01.153: pancreatic elastase | Serine Protease | Human |
| S01.155: pancreatic elastase II | Serine Protease | Human |
| S01.143: tryptase alpha | Serine Protease | Human |
| S01.131: neutrophil elastase | Serine Protease | Human |
| C02.002: calpain-2 | Cysteine Protease | Human |
| S01.211: coagulation factor XIIa | Serine Protease | Human |
| S08.090: tripeptidyl-peptidase II | Serine Protease | Human |
| S09.001: prolyl oligopeptidase | Serine Protease | Human |
| S09.004: acylaminoacyl-peptidase | Serine Protease | Human |
| T03.006: gamma-glutamyltransferase 1 | Threonine Protease | Human |
| (mammalian) | | |
| S01.214: coagulation factor IXa | Serine Protease | Human |
| S01.194: complement component 2 | Serine Protease | Human |
| C01.009: cathepsin V | Cysteine Protease | Human |
| S01.160: kallikrein hK1 | Serine Protease | Human |
| S01.236: neurosin | Serine Protease | Human |
| S01.134: myeloblastin | Serine Protease | Human |
| S01.212: plasma kallikrein | Serine Protease | Human |
| S53.003: tripeptidyl-peptidase I | Serine Protease | Human |
| C15.010: pyroglutamyl-peptidase I | Cysteine Protease | Human |
| (vertebrate) | | |
| M01.008: pyroglutamyl-peptidase II | Metallo Protease | Human |
| M54.002: archelysin (eukaryote) | Metallo Protease | Human |
| S28.002: dipeptidyl-peptidase II | Serine Protease | Human |
| S01.011: testisin | Serine Protease | Human |
| S01.257: kallikrein hK11 | Serine Protease | Human |
| S01.127: cationic trypsin (Homo sapiens- | Serine Protease | Human |
| type) | | |
| S01.258: trypsin-2 (human-type) | Serine Protease | Human |
| C01.070: dipeptidyl-peptidase I | Cysteine Protease | Human |
| S01.140: chymase (human-type) | Serine Protease | Human |
| S01.156: enteropeptidase | Serine Protease | Human |
| C01.060: cathepsin B | Cysteine Protease | Human |
| S08.072: proprotein convertase 1 | Serine Protease | Human |
| S08.073: proprotein convertase 2 | Serine Protease | Human |
| S08.074: proprotein convertase 4 | Serine Protease | Human |
| S08.075: PACE4 proprotein convertase | Serine Protease | Human |
| S01.224: hepsin | Serine Protease | Human |
| S01.162: kallikrein hK3 | Serine Protease | Human |
| S01.302: matriptase | Serine Protease | Human |
| M01.018: aminopeptidase PILS | Metallo Protease | Human |
| M01.003: aminopeptidase A | Metallo Protease | Human |
| S01.244: neuropsin | Serine Protease | Human |
| M54.950: AMZ1 g.p. (Homo sapiens) and | Metallo Protease | Human |
| similar | | |
| S01.159: prostasin | Serine Protease | Human |
| S01.223: acrosin | Serine Protease | Human |
| S01.135: granzyme A | Serine Protease | Human |
| S01.231: u-plasminogen activator | Serine Protease | Human |
| S01.232: t-plasminogen activator | Serine Protease | Human |
| S09.018: dipeptidyl-peptidase 8 | Serine Protease | Human |
| S09.019: dipeptidyl-peptidase 9 | Serine Protease | Human |
| S09.003: dipeptidyl-peptidase IV | Serine Protease | Human |
| (eukaryote) | | |
| C01.013: cathepsin X | Cysteine Protease | Human |
| C01.014: cathepsin L-like peptidase 2 | Cysteine Protease | Human |
| C01.084: bleomycin hydrolase (animal) | Cysteine Protease | Human |
| C01.018: cathepsin F | Cysteine Protease | Human and others |
| C01.032: cathepsin L | Cysteine Protease | Human and others |
| C01.034: cathepsin S | Cysteine Protease | Human and others |
| C01.040: cathepsin H | Cysteine Protease | Human and others |
| C01.035: cathepsin O | Cysteine Protease | Human and others |
| C01.037: cathepsin W | Cysteine Protease | Human and others |
| M01.013: aminopeptidase N (insect) | Metallo Protease | insect |
| C01.092: Mername-AA198 peptidase | Cysteine Protease | insects, ticks |
| M12.007: choriolysin H | Metallo Protease | japanese eel |
| M12.006: choriolysin L | Metallo Protease | japanese ricefish |
| S01.082: spermosin (Halocynthia roretzi) | Serine Protease | japanese sea squirt |
| C01.086: aminopeptidase C | Cysteine Protease | lactobacillus bacteria (cheese, |
| | | sourdough bread, kimchi) |
| C01.088: oligopeptidase E | Cysteine Protease | lactobacillus bacteria (yogurt) |
| XM12-001: meprin A complex peptidase | compound Protease | mammal |
| S01.071: kallikrein mK9 (Mus musculus) | Serine Protease | mouse |
| S01.136: granzyme B, rodent-type | Serine Protease | mouse |
| S01.163: kallikrein mK16 (Mus musculus) | Serine Protease | mouse |
| S01.164: mouse kallikrein 1 | Serine Protease | mouse |
| S01.170: 7S nerve growth factor gamma | Serine Protease | mouse |
| subunit (Mus sp.) | | |
| C01.001: papain | Cysteine Protease | papaya |
| C01.003: caricain | Cysteine Protease | papaya |
| C01.004: glycyl endopeptidase | Cysteine Protease | papaya |
| C01.002: chymopapain | Cysteine Protease | papaya, cundinamarcensis |
| | | (papaya) |
| C01.044: SmCL2-like peptidase | Cysteine Protease | parasite |
| C01.050: histolysain | Cysteine Protease | parasite |
| C01.062: cathepsin B-like peptidase | Cysteine Protease | parasite |
| (platyhelminth) | | |
| C01.076: CPA peptidase | Cysteine Protease | parasite |
| C01.077: falcipain-1 | Cysteine Protease | parasite |
| C01.098: CPC peptidase | Cysteine Protease | parasite |
| C01.075: cruzipain | Cysteine Protease | parasite - Chagas disease |
| C01.082: papain homologue (trichomonad) | Cysteine Protease | parasite responsible for a |
| | | sexually transmitted disease |
| C01.083: V-cath peptidase | Cysteine Protease | parasite, virus |
| C13.003: legumain (non-chordate) | Cysteine Protease | plant |
| C014.033: metacaspase-4 (Arabidopsis | Cysteine Protease | plant |
| thaliana) | | |
| C14.034: metacaspase-9 (Arabidopsis | Cysteine Protease | plant |
| thaliana) | | |
| M03.004: oligopeptidase A | Metallo Protease | plant |
| M17.002: leucyl aminopeptidase (plant) | Metallo Protease | plant |
| S08.092: cucumisin | Serine Protease | plant |
| S09.021: glutamyl peptidase (plant) | Serine Protease | plant |
| S14.002: peptidase Clp (type 2) | Serine Protease | plant |
| S33.001: prolyl aminopeptidase | Serine Protease | plant |
| S9G.031: leucyl endopeptidase (Spinacia | Serine Protease | plant |
| oleracea) | | |
| C13.001: legumain (plant beta form) | Cysteine Protease | Plant seed storage protein |
| | | maturation. |
| C01.041: aleurain | Cysteine Protease | plants, many types |
| C01.097: phytolacain | Cysteine Protease | pokeweed (poisinous) |
| S01.172: tonin | Serine Protease | rat |
| S01.405: kallikrein rK1 (Rattus sp.) | Serine Protease | rat |
| U9F.002: N-formylmethionyl-peptidase | Unknown | rat |
| M12.137: BHRa hemorrhagin (Bitis | Metallo Protease | snake venom |
| arietans) | | |
| M12.151: ecarin | Metallo Protease | snake venom |
| M12.153: fibrinolytic peptidase (Philodryas | Metallo Protease | snake venom |
| olfershii) | | |
| M12.169: metallopeptidase (Bothrops | Metallo Protease | snake venom |
| moojeni) | | |
| S01.180: platelet-aggregating venom | Serine Protease | snake venom |
| peptidase | | |
| S01.428: LV-Ka peptidase | Serine Protease | snake venom |
| S9G.025: snake venom coagulation factor X | Serine Protease | snake venom |
| activator, serine-type (Bungarus fasciatus | | |
| {Cerastes vipera}, {Ophiophagus hannah}) | | |
| S9G.027: scutelarin (Oxyuranus scutellatus) | Serine Protease | snake venom |
| C01.067: insect 26/29 kDa peptidase | Cysteine Protease | tsetse fly |
| M9A.010: aminopeptidase yscCo-II | Metallo Protease | unknown |
| M9E.002: alanine carboxypeptidase | Metallo Protease | unknown |
| S9G.073: intestinal Arg-specific | Serine Protease | unknown |
| endopeptidase | | |
| C03.013: rhinovirus 14 3C peptidase | Cysteine Protease | virus (common cold) |
| C01.085: bleomycin hydrolase (yeast) | Cysteine Protease | yeast |
| M01.007: Aap1′ aminopeptidase | Metallo Protease | yeast |
| S01.276: yeast-lytic peptidase (Rarobacter) | Serine Protease | yeast |
| C01.068: vitellogenic cathepsin B | Cysteine Protease | yellow fever mosquito |

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. The term “and/or” is used in the same manner, meaning “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

To the extent that the phrase “one or more of, A, B, and C” is employed herein, (e.g., a data store configured to store one or more of, A, B, and C) it is intended to convey the set of possibilities A, B, C, AB, AC, BC, and/or ABC (e.g., the data store may store only A, only B, only C, A&B, A&C, B&C, and/or A&B&C). It is not intended to require one of A, one of B, and one of C. When the applicants intend to indicate “at least one of A, at least one of B, and at least one of C”, then the phrasing “at least one of A, at least one of B, and at least one of C” will be employed.

What is claimed is:

1. A chemical exchange saturation transfer (CEST) contrast agent, comprising:
a ligand; and
a functional group linked to the ligand, the functional group having a hydrogen exchange site and being capable of undergoing a change in chemical functionality by enzyme catalysis or reaction with a metabolite so as to change the chemical exchange rate or the MR frequency of the hydrogen exchange site.

2. The contrast agent of claim 1, where the ligand is selected from the group consisting of N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecane-N,N′,N″-triacetic acid (DO3A); and derivatives thereof.

3. The contrast agent of claim 2, comprising a lanthanide metal ion chelated with the ligand, the lanthanide metal ion being capable of shifting the MR frequency of the functional group to unique frequencies to facilitate selective detection.

4. The contrast agent of claim 3, where the lanthanide metal ion is selected from the group consisting of Eu³⁺, Tm³⁺, and Yb³⁺.

5. The contrast agent of claim 4, where the functional group is selected from the group consisting of an imine, an amine, an amide, a hydroxyl, a thiol, and a phosphate.

6. The contrast agent of claim 5, where the ligand is linked to a nanocarrier.

7. The contrast agent of claim 6, where the nanocarrier is selected from the group consisting of a monomer, a polymer, a dendrimer, a pegylated polylysine, and a liposome.

8. The contrast agent of claim 7, where the polymer is a hydroxylaminepropylmethacrylate polymer.

9. The contrast agent of claim 7, where a second contrast agent is entrapped within the liposome core.

10. The contrast agent of claim 5, comprising a target-specific ligand linked to an amide functional group, the target-specific ligand capable of being cleaved by an enzyme to convert the amide functional group to an amine.

11. The contrast agent of claim 10, where the enzyme is caspase-3, MMP-2, MMP-9, Cathepsin B, or esterase.

12. The contrast agent of claim 10, where the target specific ligand is a peptide.

13. The contrast agent of claim 12, where the peptide is DEVD (SEQ ID NO 1).

14. The contrast agent of claim 5, the functional group to react in the presence of a crosslinking enzyme to link a new substituent to the functional group to produce a detectable change in the CEST effect.

15. The contrast agent of claim 14, where the crosslinking enzyme is glutaminase.

16. The contrast agent of claim 15, where the functional group is an amine and the new substituent is an aliphatic amide of glutamine.

17. The contrast agent of claim 5, where the functional group is a hydroxyl group to be phosphorylated in the presence of kinase enzymes to produce a detectable change in the CEST effect.

18. The contrast agent of claim 5, where the functional group is an aminoanilide group capable of reacting with NO to produce a detectable change in the CEST effect.

19. The contrast agent of claim 18, where the aminoanilide group is to react with NO to form a triazene product.

20. A compound, comprising:
a ligand with an MR-sensitive peptide sequence to be modified by an enzyme.

21. The contrast agent of claim 20, where the ligand is selected from the group consisting of N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecane-N,N′,N″-triacetic acid (DO3A); and derivatives thereof.

22. The compound of claim 21, the MR-sensitive peptide sequence being DEVD (SEQ ID NO 1).

23. The compound of claim 21, the MR-sensitive peptide sequence being a sequence to be covalently modified by an enzyme.

24. The compound of claim 21, the enzyme being caspase-3.

25. The compound of claim 21, the enzyme being one of an enzyme to cleave a peptide sequence, and an enzyme to covalently modify a peptide sequence.

26. The compound of claim 21, the peptide being a molecular entity that possesses a hydrogen atom that exchanges with a hydrogen atom of a solvent molecule at a rate slower than the difference in MR chemical shifts for the hydrogens of the molecules.

27. A compound, comprising:
DOTA with an MR-sensitive chemical functional group to be modified by a CEST-altering-molecule.

28. The compound of claim 27, the CEST-altering-molecule being one of, a catalyst, and a reactant.

29. A method, comprising:
introducing an agent to a cell, tissue, or patient, the agent being configured to selectively produce a CEST effect, the agent including a chemical functional group to be modified by a CEST-altering molecule;
applying one or more RF pulses to the cell, tissue, or patient, the RF pulses to produce an MR signal in the cell, tissue, or patient;
acquiring the MR signal; and
producing one or more images from the MR signal, the one or more images illustrating a change in the CEST effect produced by the CEST-altering molecule.

30. The method of claim 29, the agent to facilitate detecting one or more of, an enzyme, a biomarker, and a member of Protease Set A.

31. The method of claim 29, the agent being a contrast agent having one or more protons available to exchange into water, where an exchange of protons between the agent and water can be selectively controlled by an RF pulse in an MR imaging sequence.

32. The method of claim 29, where the agent is linked to a nanocarrier.

33. The method of claim 29, the method including introducing an unresponsive agent with the agent configured to selectively produce a CEST-effect.

34. The method of claim 29, where the agent configured to selectively produce a CEST-effect is linked to at least a second agent with a unique PARACEST frequency to selectively monitor activity of one or more of, an enzyme, and biomarkers.

35. An MRI apparatus, comprising:
a logic to produce one or more RF pulses;
a logic to receive an MR signal; and
a logic to produce an image from the MR signal,
the one or more RF pulses being configured to selectively detect an altering of a CEST affect produced by an agent administered to a cell, tissue or patient and subjected to the one or more RF pulses, the agent being configured to selectively produce a CEST effect, the agent including a chemical functional group that can be cleaved off by a CEST-altering molecule.
